(19) United States
(12) Patent Application Publication
(10) Pub. No.: US 2005/0163829 A1
(43) Pub. Date: Jul. 28, 2005

(54) Device and patch for controlled release of vanillin

(76) Inventor: Norbert Hoenzelaer, Kleve-Rindern (DE)

Correspondence Address: LERNER AND GREENBERG, PA, P O BOX 2480, HOLLYWOOD, FL, 33022-2480, US

(21) Appl. No.: 11/084,349

(22) Filed: Mar. 18, 2005

Related U.S. Application Data
(63) Continuation of application No. PCT/EP02/13820, filed on Dec. 6, 2002.

(30) Foreign Application Priority Data
Dec. 7, 2001 (DE) 101 60 110.7

(51) Int. Cl. A61L 15/16; A61F 13/02

(52) U.S. Cl. 424/448

(57) ABSTRACT

The invention relates to devices for continuous release of at least one gaseous vanillin compound, comprising a layer impermeable to the gaseous vanillin compound; a layer permeable to the gaseous vanillin compound, which is localized between said two layers. The invention also relates to a method for the production of said devices, to the devices obtained according to said method, to the utilization of the above-mentioned devices, for stimulating the release of serotonin in the central nervous system and to the utilization of said devices in a diet plan.

### CROSS-REFERENCE TO RELATED APPLICATION

This is a continuing application, under 35 U.S.C. § 120, of co-pending international application No. PCT/EP02/13820, filed Dec. 6, 2002, which designated the United States; this application also claims the priority, under 35 U.S.C. § 119, of German patent application No. 10160110.7, filed Dec. 7, 2001; the prior applications are herewith incorporated by reference in their entirety.

### BACKGROUND OF THE INVENTION

1. Field of the Invention

The present invention relates to a device for the controlled release of vanillin, ethylvanillin derivatives or mixtures thereof, to a process for producing said device, to a device obtainable by said process, to the use of a composition comprising vanillin, ethylvanillin or mixtures thereof, and to the use of the device.

Numerous aromas develop pronounced effects on various receptors of neurotransmitters (GABA, NMDA, KA, nACh). Fragrances such as geraniol (“rose”) and butyl acetate (“fruit”) stimulate the GABA receptor. In high doses, receptor stimulants of this kind even have a hypnotic and anesthetizing effect. Vanillin, by contrast, similarly to caffeine, inhibited said receptor. Since, according to current understanding, the most important physiological effects of caffeine are based on this inhibition, vanillin could have a similar stimulant effect to caffeine. Vanillin also inhibited the nicotinic acetylcholine receptor (nACh), a property which it shares with the snake poison cobratoxin or the arrow poison curare. Vanillin also had an inhibitory effect on the glutamate receptors (NMDA, KA). This effect is comparable in principle with that of phencyclidine, which is also known as a narcotic (“Angel dust”).

As well as this receptor inhibition, in the central nervous system vanillin also influences the craving to consume food. In this context it is supposed that the vanillin is able to increase the concentration of the neurotransmitter serotonin in the brain. Increased brain serotonin concentration, however, leads demonstratively to a reduced craving to consume food.

Since serotonin cannot be prepared synthetically, and since there is in any case virtually no possibility of uptake of said neurotransmitter into the brain, owing to the compartment systems located there, attempts have now been made to obtain an increase in brain serotonin concentration through the use of the fragrance vanillin.

Uptake of this fragrance via the blood-brain barrier is possible on account of its lipophilic properties. A second possible uptake pathway, besides that of the blood-brain barrier, is the olfactory nerve (bulbus olfactorius), which allows transneural transport into the brain (Neurotoxicology and Teratology 1990/12/pp. 445-9). Vanillin, accordingly, is able to enter the central nervous system via the nose. In the patent literature, accordingly, devices have been numerously described by means of which this fragrance can be vaporized in the vicinity of the body and so breathed in continuously (uptake of vanillin via the olfactory nerve) or with which vanillin can be supplied continuously to the circulation (uptake via the blood-brain barrier).

For instance, EP A 844 872 (corresponds to WO 97/03658) describes a device for administering vanillin that comprises lint or a similar material impregnated with the vanillin-releasing substance, the lint being contained in a self-adhesive patch for application to the skin, and the exposed surface of the patch having a central hole through which the vanillin is able to emerge from the lint. The disadvantage of this device, however, is that the rate at which the vanillin emerges cannot be adequately regulated.

EP A 645 081 describes an insect control device comprising a container which is covered by a film impervious to gaseous essential oils. Following removal of a protective layer, the insecticidal compositions contained in said container emerge from the container continuously through the film. The disadvantage of these containers is that they are not suitable for continuous release of vanillin in the vicinity of the body.

### SUMMARY OF THE INVENTION

The object on which the present invention is based was to overcome the disadvantages arising from the prior art.

The object of the invention, moreover, was to provide a device by means of which it is possible to release vanillin continuously in the vicinity of the body so that it can be taken up via the nose.

A further object of the invention was to provide a device with which vanillin can be taken up continuously via the skin.

The above objects have been achieved by means of a device for continuously releasing at least one vanillin compound such as vanillin itself (3 methoxy-4 hydroxybenzaldehyde) or at least one vanillin derivative, preferably ethylvanillin (3 ethoxy-4 hydroxybenzaldehyde), or mixtures thereof, comprising a layer impermeable to the at least one gaseous vanillin compound, a layer permeable to the at least one gaseous vanillin compound, and a composition comprising the at least one vanillin compound which is located between these two layers. The device is used in the vicinity of the body of a wearer, preferably by adhesion to the skin of the wearer. The wearer is preferably a mammal such as a human being, a dog, a pig, a hamster, a cat, a hare or a rabbit, a human being particularly preferred.

The layer impermeable to the at least one gaseous vanillin compound is preferably a film comprising polyester, polypropylene, polyethylene, ethylene-propylene copolymers, polyamides, such as nylon 6,6, metals, such as aluminum, polyethylene, poly(tetrafluoroethylene), polycarbonate, polyethylene terephthalate, polybutyrate, polyurethane or polyvinyl chloride, particular preference being given to a film comprising polypropylene or polyethylene or to a film comprising an ethylene-propylene copolymer. Among these films, very particular preference is given to polyethylene films which are obtainable under the commercial designation Sclairfilm® from DuPont, Canada. The thickness of the layer impermeable to the at least one gaseous vanillin compound is preferably chosen so that within a time interval of 24 hours not more than 1%, preferably not more than 0.1%, and more preferably not more than 0.01% by weight of the composition can evaporate via this film at room temperature under a pressure of 1 bar. Preferably this layer has a thickness in a range from 0.001 to 1 mm, more preferably in a range from 0.01 to 0.5 mm, and with further preference in a range from 0.1 to 0.25 mm.

The layer permeable to the at least one gaseous vanillin, gaseous vanillin derivative or mixtures thereof is preferably a film that has the property whereby within a time interval of 24 hours at least 10%, preferably at least 25%, and more preferably at least 50% by weight of the vanillin, ethyl vanillin or mixtures thereof that are present in the composition can evaporate via this film at room temperature under a pressure of 1 bar. Preferred films are films comprising polyethylene, polyamide, ethylene-vinyl acetate copolymer or mixtures of these monomers. The permeable layer preferably has a thickness in a range from 0.001 to 1 mm, more preferably in a range from 0.01 to 0.5 mm, and with further preference in a range from 0.1 to 0.25 mm. It is further preferred in this context that via the choice of material of the permeable layer and its thickness the amount of the composition evaporated through this layer in a defined time interval at a defined temperature and under a defined pressure can be regulated. In one particular embodiment of this device the permeable layer has microscopic holes having a diameter in a range between 1 and 100 Å, more preferably in a range between 10 and 50 Å, which is preferably obtainable by bombarding the layer with heavy atoms, such as with barium atoms, for example. The density of holes in the film, indicated in the number of holes per mm², and the diameter of the holes likewise make it possible to regulate the amount of composition evaporated through the permeable layer within a defined time interval at a defined temperature and under a defined pressure.

It is further preferred in this context for both the permeable layer and the impermeable layer to have a thickness of not more than 0.1 mm, more preferably of not more than 0.01 mm, so that the device of the invention is not rigid and is able when placed onto an uneven surface to conform to that surface.

Other features which are considered as characteristic for the invention are set forth in the appended claims.

Although the invention is illustrated and described herein as embodied in a device and patch for controlled release of vanillin, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 shows an embodiment of the device of the invention as a cross section, in which the impermeable layer is shaped in the form of a vessel. With this device, continuous uptake of the at least one vanillin compound nasally or pulmonarily is possible.

FIG. 2 shows an embodiment of the device of the invention as a cross section, in which the impermeable layer is shaped in the form of a vessel. With this device, continuous uptake of the at least one vanillin compound trans-dermally is possible.

FIG. 3 shows an embodiment of the device of the invention in the form of a patch as a cross section. With this patch, continuous uptake of the at least one vanillin compound nasally or pulmonarily is possible.

FIG. 4 shows an embodiment of the device of the invention in the form of a patch as a cross section. With this patch, continuous uptake of the at least one vanillin compound trans-dermally is possible.

### DESCRIPTION OF THE PREFERRED EMBODIMENTS

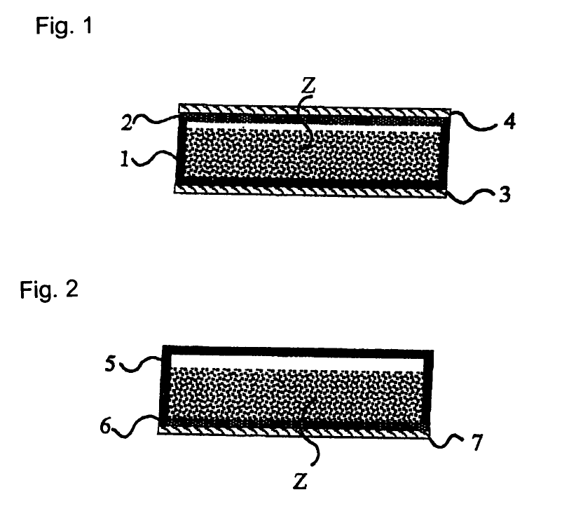

With reference to FIG. 1, in one embodiment of the device of the invention it comprises
the layer impermeable to the at least one gaseous vanillin compound, this layer having the form of a container (1),
the layer permeable to the at least one gaseous vanillin compound, as top layer (2), the top layer (2) adhering to the container (1) with sufficient strength that a force F₁ of at least 0.5 N, preferably of at least 1 N, and more preferably of at least 5 N is required to separate the top layer (2) from the container (1),
and the side of the container (1) opposite the top layer (2) being provided, on its side facing away from the top layer (2), with an adhesive,
a first, protective liner layer (3), which attaches adhesively to the side of the container (1) that has been provided with the adhesive, and which can be separated from the container (1) by peeling by hand, preferably through the action of a force of less than 10 N, more preferably of less than 1 N, and with further preference of less than 0.1 N, the adhesive remaining on the container (1),
a second protective liner layer (4), made of a material which is impermeable to gaseous vanillin or to gaseous vanillin derivatives or to gaseous mixtures thereof, which attaches adhesively to the side of the top layer (2) that is facing away from the container (1), and can be separated from the top layer (2) by the action of a force F₂, which is smaller than the force F₁, and
the composition (Z) in the container (1), said composition being free from active insecticidal substances and comprising vanillin or vanillin derivatives or mixtures thereof.

When this device, following removal of the first protective layer (3), is adhered to the skin of the wearer, the impermeable container (1) entering into contact with the skin of the wearer, it allows the continuous uptake of vanillin or vanillin derivatives or mixtures thereof through the nose (nasal uptake) or the lungs (pulmonary uptake) of the wearer. This is made possible by virtue of the fact that, owing to the body temperature of the wearer, the evaporation of the composition in the container and hence also the passage of the at least one gaseous vanillin compound through the top layer (2), after the second protective liner layer (4) has been removed, is promoted.

In the case of the above-described embodiment of the devices of the invention, the container (1) is preferably transparent. It is further preferred for the container (2) to have the form of a trough whose open side possesses a flat, outwardly curved edge. In this case the top layer (2) is attached to the flat, outwardly curved edge of the transparent trough. In connection with this preferred embodiment it is further preferred for the device, besides the optionally transparent, trough-shaped container (1), the top layer (2) and the protective layers (3) and (4), to comprise a paper board packaging which is folded so that the paper board is two-ply and between these two plies in the two-ply region, accommodates the flat, outwardly bent edges and the top layer (2) and also the protective layer (4) or (3), respectively, while the transparent trough is arranged visibly and transparently outside this packaging. In the case of a transparent container (1) the latter is composed preferably of polyethylene or polypropylene. The advantage when using a transparent container is that the amount of composition still present in the container can be determined at any time for the wearer. The packaging serves preferably as an advertising space.

With reference to FIG. 2, in another embodiment of the device of the invention it comprises
the layer impermeable to the at least one gaseous vanillin compound, which has the form of a container (5),
the layer permeable to the at least one gaseous vanillin compound, as top layer (6),
the top layer (6) adhering to the container (5) with sufficient strength that a force F₁ of at least 0.5 N, preferably of at least 1 N, and more preferably of at least 5 N is required to separate the top layer (6) from the container (5),
and the top layer (6) being provided on the side facing away from the container (5) with an adhesive,
a protective liner layer (7), made of a material which is impermeable to gaseous vanillin or to gaseous vanillin derivatives or to gaseous mixtures thereof, which attaches adhesively to the side of the top layer (6) provided with the adhesive and can be separated from the top layer (6) by the action of a force F₂, which is smaller than the force F₁, preferably by peeling by hand, the adhesive remaining on the top layer (6),
the composition (Z) in the container (5), said composition being free from active insecticidal substances and comprising the at least one gaseous vanillin compound.

When this device, following the removal of the protective layer (7) is adhered to the skin of the wearer, the permeable top layer (6) entering into contact with the skin of the wearer, it allows the continuous, trans-dermal uptake of vanillin or vanillin derivatives or mixtures thereof through the wearer.

The container (1) or (5) preferably has a length x and a width y in a range from 0.5 to 20 cm, more preferably in a range from 1 to 10 cm, and with further preference in a range from 2 to 5 cm, and a height z in a range from 0.01 to 0.5 cm, more preferably in a range from 0.1 to 0.4 cm, and with further preference in a range from 0.2 to 0.3 cm. It is further preferred for the length x and the width y of the patch to be greater by a factor of at least 2, preferably at least 5, and more preferably at least 10, than the height z of the container (1) or (5) respectively. It is further preferred for the capacity of the container (1) or (5) to be in a range from 0.0025 to 200 cm³, more preferably in a range from 0.01 to 10 cm³, and with further preference in a range from 0.1 to 1 cm³.

Preferably, the top layer (2) or (6) attaches to the container (1) or (5), respectively, with sufficient strength that it cannot be grasped with the hand or torn from the container by pulling with the hand.

The difference in adhesion between the top layer (2) or (6) and the container (1) or (5), on the one hand, and between the protective liner film (4) or (7) and the top layer (2) or (6), on the other hand, which results in the force F₂ required to tear the protective film from the top layer being smaller than the force F₁ required to tear the top layer from the container, is made possible preferably through the use of adhesives which have different bond strengths or else by means of areas differing in size in which adhesion takes place between the individual components.

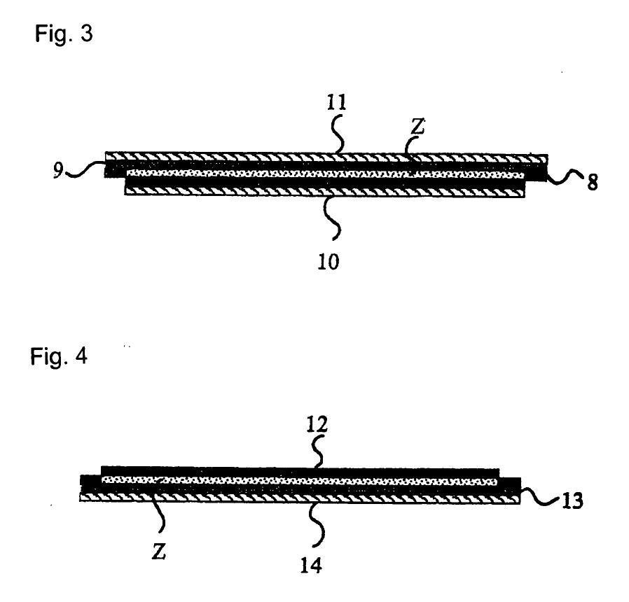

With reference to FIG. 3, in another preferred embodiment of the device of the invention it is shaped as a patch and comprises
the layer (8) impermeable to the at least one gaseous vanillin compound,
the overlying layer (9), permeable to the at least one gaseous vanillin compound, and the composition (Z) located between both layers,
layer (8) impermeable to the at least one gaseous vanillin compound being provided on its side facing away from the permeable layer (9) with an adhesive,
a first, protective liner layer (10), which attaches adhesively to the side of the impermeable layer (8) that has been provided with the adhesive, and which can be separated from this film by peeling by hand, preferably through the action of a force of less than 10 N, more preferably of less than 1 N, and with further preference of less 0.1 N, the adhesive remaining on the impermeable layer (8),
a second protective liner layer (11), made of a material which is impermeable to the at least one gaseous vanillin compound, which attaches adhesively to the permeable layer (9) on its side that is facing away from the impermeable layer (8), and can be separated by peeling by hand, preferably by the action of a force of less than 10 N, more preferably less than 1 N and with further preference less than 0.1 N, from the permeable layer (9).

When this patch is brought into the vicinity of the body, by means for example of the adhering of said patch to the skin of the wearer, following the removal of the first protective layer (10), the impermeable layer (8) entering into contact with the skin, it permits the continuous evaporation of the composition and hence the uptake of the at least one gaseous vanillin compound, through the nose or the lungs of the wearer. This is made possible by virtue of the fact that, owing to the body temperature of the wearer, the evaporation of the composition in the patch and hence also the passage of the at least one gaseous vanillin compound, through the permeable layer (9), after the second protective liner layer (11) has been removed, is promoted.

With reference to FIG. 4, in a further embodiment of the device of the invention it is likewise designed as a patch and comprises
the layer (12) impermeable to the at least one gaseous vanillin compound,
the underlying layer (13), permeable to the at least one gaseous vanillin compound, and the composition (Z) located between both layers,
the layer (13) permeable to the at least one gaseous vanillin compound, being provided on its side facing away from the impermeable layer (12) with an adhesive,
a protective liner layer (14), which attaches adhesively to the side of the permeable layer (13) that has been provided with the adhesive, and can be separated from this layer by peeling by hand, preferably by the action of a force of less than 10 N, more preferably of less than 1 N and with further preference of less than 0.1 N, the adhesive remaining on the permeable layer (13).

When this patch is brought into the vicinity of the body, by means for example of said patch being adhered to the skin of the wearer, following the removal of the protective layer (15), the permeable layer entering into contact with the skin, it allows the continuous trans-dermal uptake of the at least one gaseous vanillin compound, through the wearer.

If the device of the invention is designed in the form of a patch, then it is preferred for this patch not to comprise any lint or similar materials impregnated with the composition. In this context it is further preferred for the composition to have a dynamic viscosity in range from 10² to 10⁶, more preferably in a range from 10³ to 10⁵ mPa·s at 20° C. in accordance with DIN 53211.

The adhesive present in the device of the invention preferably comprises those adhesives known to the skilled worker that can be used to attach a patch to the skin of a wearer. Preferred adhesives are those which are offered by Dow Corning Corporation, USA, under the commercial designation BIO PSA, and also acrylate-based or silicone-based adhesives which are approved for medical use.

The protective liner layers are preferably films which comprise the materials listed in connection with the impermeable layer. The thickness of these protective layers as well corresponds preferably to that of the permeable or impermeable layer. Particularly preferred protective layers used are films comprising polyethylene or polypropylene.

The purpose of the protective layers attaching adhesively to the permeable layer is to prevent evaporation of the composition from the device before it is used for the controlled release of the at least one gaseous vanillin compound. These protective layers must, accordingly, be impermeable to the at least one gaseous vanillin compound. For this reason they preferably have a thickness such that not more than 1%, preferably not more than 0.1%, and more preferably not more than 0.01% by weight of the composition is able to evaporate via this protective layer within a time interval of 24 hours at room temperature under a pressure of 1 bar.

The composition comprised in the device of the invention is based preferably on
(α1) the at least one gaseous vanillin compound, in an amount in a range from 0.1% to 100% by weight, preferably in a range from 1% to 50% by weight, and with further preference in a range from 5% to 20% by weight,
(α2) a vehicle in an amount in a range from 0% to 99% by weight, more preferably in a range from 20% to 70% by weight, and with further preference in an amount in a range from 40% to 60% by weight, and
(α3) excipients in an amount in a range from 0% to 50% by weight, more preferably in an amount in a range from 10% to 40% by weight, and with further preference in an amount in a range from 20% to 30% by weight,
   the sum of components (α1) to (α3) being 100% by weight.

The term “vanillin compound” is used to refer to the chemical substance vanillin, 3-methoxy-4-hydroxybenzaldehyde and its isomers and derivatives in which one or more hydrogen atoms are substituted by nitro groups or lower alkyl groups having one to three carbon atoms. Preferred vanillin compounds are vanillin, ethylvanillin, 3-ethoxy-4-methoxybenzaldehyde, and isovanillin, 4-methoxy-3-hydroxybenzaldehyde.

The vehicle preferably comprises monohydric alcohols, such as ethanol, polyhydric alcohols, such as glycerol, ethylene glycols or their ether derivatives, such as diethylene glycol, polyethylene glycol, diethylene glycol diethyl ether, polyethylene glycol dimethyl ether, essential oils, examples being cedar wood oil, cedar leaf oil, thuja oil, lavender oil, lavandin oil, lemon oil, Litsea cubeba oil (may chang oil) or verty oil, silicone oils, waxes, fats, such as olive oil, groundnut oil or coconut fat, or mixtures of at least two of the aforementioned vehicles. The vehicle can further comprise resinous and rubbery materials, such as methylcellulose, ethylcellulose, hydroxypropyl methylcellulose, natural rubber, butadiene—styrene copolymer rubber, and ethylene-propylene copolymer rubber.

Excipients present are preferably substances which promote the evaporation of the the at least one gaseous vanillin compound. Preferred evaporation-promoting excipients are selected from the group consisting of hexyl acetate, isobutyl acetate, cyclohexyl acetate, prenyl acetate, isononyl acetate, isobornyl acetate, linalyl acetate, benzyl acetate, ethyl butyrate, camphor, ethyl caproate, methyl caproate, carenene, limonene, the terpene oil from oranges, allyl heptanoate, methyl hexenoate, ethyl isobutyrate, methyl pentyl ketone, myrcene, phellandrene, pinene, butyl valerate, terpinolene, liquid paraffins or mixtures of at least two of these compounds. Preferred excipients are, furthermore, dyes, preservatives, viscosity regulators, UV stabilizers, and further aromatic compounds, coumarin for example. Further-preferred excipients are fragrances other than the at least one gaseous vanillin compound, preferably selected from the group consisting of orange, neroli, jasmine, balm, sandalwood, bergamot, oak moss, eucalyptus, fennel, camomile, caraway, sage, rosemary, star anise, tea tree oil, incense, cinnamon bark, stone pine, almond oil, jojoba or mixtures of at least two thereof. If the composition is used in a device for transdermally administering the at least one gaseous vanillin compound, then the composition preferably also includes, as excipients, penetration promoters, examples being mixtures of 1 menthol and propylene glycol.

In one preferred embodiment of the above-described device the composition comprises as excipient compounds in which the at least one gaseous vanillin compound may be included. Excipients preferred in this context are cyclodextrins, examples being α, β or γ cyclodextrins, activated carbon, or zeolites. In such a case these excipients and the at least one gaseous vanillin compound are present in a molar ratio in a range from 5:1 to 1:5, more preferably in a range from 2:1 to 1:2, and very preferably in a molar ratio of 1:1. It is further preferred, in one embodiment according to the invention, for the excipients serving as vehicles to be modified in such a way that the at least one gaseous vanillin compound is or are delivered in retarded fashion. Particularly suitable for this purpose is a solid vehicle such as cellulose and liquid substance absorbed by this solid vehicle, such as water or alcohol, which is delivered over a relatively long period of time, and the vanillin or vanillin derivative or mixture thereof, admixed where appropriate with a solubilizer such as a surfactant to the liquid substance, is delivered in a retarded fashion in comparison to a system without this excipient combination. The preparation of the at least one gaseous vanillin compound encapsulated in these excipients is accomplished preferably by contacting the individual components with one another in a suitable solvent, ethanol for example, or else by mixing the components with one another in the dry state, preferably by kneading, or slurrying in small amounts of a solvent, preferably in small amounts of water. The at least one gaseous vanillin compound is or are released preferably by the destruction of the resultant microcapsule structures by the action of mechanical force, preferably by pressure, which then allows the at least one gaseous vanillin compound to evaporate. The major advantage of encapsulating the at least one vanillin compound is that its release can be restricted to particular areas through application of a pressure, by pressing with the finger, for example. In this way it is possible to ensure the release of the at least one gaseous vanillin compound over a relatively long period of time.

As the composition it is preferred to use a mixture of floral vanillin (ref. C6031/E Flora from A. Algto Ltd) as the at least one vanillin compound and ethanol. In order to increase the viscosity it is possible for this mixture further to comprise viscosity regulators, glycol for example.

It is further preferred for at least 50%, preferably at least 75%, and more preferably at least 99% of the capacity of the container (1) to be filled with the composition. In a particularly preferred embodiment, the composition suitable for the device of the invention shaped as a patch is formulated with 1-5% by weight of the vanillin compound, 10-30% by weight synthetic rubber Ecomelt T8 EX 24 (Source: Colano Co., Switzerland), 50-75% by weight synthetic rubber Durotak H 112 (Source: National Starch Co., USA), 1-5% by weight ethylcellulose N50 NF, 1-5% by weight glycerol, and 1-5% propylene glycol, provided that the total of al ingredients is 100% by weight.

The invention also relates to a process for producing the above-described device for controlledly releasing the at least one gaseous vanillin compound.

If the impermeable layer is shaped in the form of a container then the device of the invention is produced preferably by
introducing the above-described composition into the above-described container,
and subsequently sealing the container with the impermeable layer in such a way that separation of the impermeable layer from the container requires a force of at least 0.5 N, preferably of at least 1 N, and more preferably of at least 5 N.

If the container, owing to the thickness of the impermeable layer, is not rigid, then the container can first be inserted into a corresponding mold. Subsequently the composition is introduced into the container in the mold.

If the device is shaped in the form of a patch, then the device of the invention is produced preferably by introducing the composition between the permeable layer and the impermeable layer. This is preferably accomplished by first applying the composition to a defined area of the permeable or impermeable layer and placing the other layer over the composition, the permeable and impermeable layers being connected to one another at those points at which they directly contact one another. This joining takes place preferably by welding or adhesive bonding.

The invention further relates to the devices obtainable by processes described above.

The invention also relates to the use of the at least one gaseous vanillin compound, to regulate the craving to consume food.

The present invention also provides for the use of an above-described device for the trans-dermal, nasal or pulmonary uptake of the at least one gaseous vanillin compound, to stimulate the release of serotonin in the central nervous system.

The invention relates, moreover, to the use of the at least one gaseous vanillin compound, taken up trans-dermally, for producing a treatment means, preferably a medicinal product, for regulating the craving to consume food.

The present invention further provides for the use of an above-described device for producing a treatment means, preferably a medicinal product, for the trans-dermal, nasal or pulmonary uptake of the at least one gaseous vanillin compound, to stimulate the release of serotonin in the central nervous system.

The invention further relates to the use of the at least one gaseous vanillin compound, taken up trans-dermally, as a treatment means, preferably a medicinal product, for regulating the craving to consume food.

The present invention also provides for the use of an above-described device as a treatment means, preferably a medicinal product, for trans-dermal, nasal or pulmonary uptake of the at least one gaseous vanillin compound, to stimulate the release of serotonin in the central nervous system.

Finally the present invention also relates to the use of an above-described device in a dietary regime or in combination with foods selected for a particular diet. For example, the device of the invention is used together in a food-combining diet in combination with foods suitable for the diet and containing only protein or carbohydrates. In another diet, the device of the invention is used with liquid food such as fruit and vegetable juices.

The invention is now illustrated with reference to non-limiting examples.

### EXAMPLES

### Example 1

Introduced into the virtually transparent, trough-shaped container depicted in FIG. 1, comprising a polypropylene film 0.1 mm thick and having a base area of 14 cm² and a height of 5 mm, were 6.5 ml of a composition made up of 50% by weight floral vanillin and 50% by weight ethanol. Subsequently a polyethylene film having a thickness of 200 μm was bonded to the outwardly curved edge of the trough-shaped container. A polypropylene protective layer 280 μm thick was provided with an adhesive, one corner of the protective layer remaining free of adhesive, in order to allow the film to be peeled off by hand. Subsequently the protective film was bonded to the polyethylene film. Adhesive was likewise applied to the underside of the container. The device thus obtained was subsequently adhered to the surface of the hand. Following the removal of the protective layer, there was continuous evaporation of ethyl vanillin via the permeable layer.

### Example 2

Introduced into the virtually transparent, trough-shaped container depicted in FIG. 1, comprising a polypropylene film 0.1 mm thick and having a base area of 14 cm² and a height of 5 mm, were 6.5 ml of a composition made up of 50% by weight floral vanillin and 50% by weight ethanol. Subsequently a polyethylene film having a thickness of 200 μm was bonded to the outwardly curved edge of the trough-shaped container and subsequently an adhesive was applied to the surface of this film. The resulting device was subsequently adhered to the surface of the hand. Following the removal of the protective layer, there was continuous trans-dermal uptake of ethyl vanillin.

In both examples, in the case of a group of 10 adult test persons having a body mass index of between 25 and 27, over a wearing period of 45 days per unit, a reduction in food consumption was observed over a period of 31 days. The test persons lost on average about 0.4 kg per day.

This application claims the priority, under 35 U.S.C. § 119, of German patent application No. 10160110.7, filed Dec. 7, 2001; the entire disclosure of the prior application is herewith incorporated by reference.

1. A device in the form of a patch for continuously releasing at least one gaseous vanillin compound, comprising a layer impermeable to said at least one gaseous vanillin compound, a layer permeable to said at least one gaseous vanillin compound, and a composition comprising at least one vanillin compound, said composition being located between these two layers, the device not comprising lint impregnated with the composition.

2. The device as claimed in claim 1, wherein said at least one vanillin compound is selected from the group consisting of vanillin and ethylvanillin.

3. The device as claimed in claim 1, wherein the impermeable layer is provided with an adhesive.

4. The device as claimed in claim 1, further comprising a first, protective liner layer attached adhesively to the side of said impermeable layer, and a second protective liner layer, which is impermeable to the at least one gaseous vanillin compound, attached adhesively to the permeable layer on its side that is facing away from the impermeable layer.

5. The device as claimed in claim 1, wherein the composition has a dynamic viscosity in a range from 10² to 10⁶ mPa·s at 20° C. in accordance with DIN 53211.

6. The device as claimed in claim 5, wherein the composition has a dynamic viscosity in a range from 10³ to 10⁵ mPa·s at 20° C. in accordance with DIN 53211.

7. The device as claimed in claim 1, wherein the composition is based on
(α1) vanillin and/or ethyl vanillin in an amount in a range from 0.1% to 100% by weight;
(α2) a vehicle in an amount in a range from 0% to 99% by weight; and
(α3) excipients in an amount in a range from 0% to 50% by weight;
the sum of components (α1) to (α3) being 100% by weight.

8. The device as claimed in claim 7, wherein said vehicle comprises synthetic rubber.

9. The device as claimed in claim 1, wherein the layer impermeable to the at least one gaseous vanillin compound has a thickness such that within a time interval of 24 hours not more than 1% by weight of the composition can evaporate via this film at room temperature under a pressure of 1 bar.

10. The device as claimed in claim 9, wherein said layer has a thickness in a range from 0.001 to 1 mm.

11. The device as claimed in claim 1, wherein the layer permeable to the at least one gaseous vanillin compound has the property whereby within a time interval of 24 hours at least 10%, by weight of the at least one vanillin compound that are present in the composition can evaporate at room temperature under a pressure of 1 bar.

12. The device as claimed in claim 11, wherein said permeable layer comprises a film comprising polyethylene, polyamide, ethylene-vinyl acetate copolymer or mixtures thereof.

12. The device as claimed in claim 11, wherein said permeable layer has a thickness in a range from 0.001 to 1 mm,

13. The device as claimed in claim 1, wherein the permeable layer comprises a film having holes having a diameter in a range between 1 and 100 Å.

14. The device as claimed in claim 1, wherein the permeable layer comprises a film having holes having a diameter, and a density, such that within a time interval of 24 hours at room temperature under a pressure of 1 bar at least 10% by weight of the vanillin and/or ethyl vanillin present in the composition is able to evaporate.

15. The device as claimed in claim 14, wherein the film has holes with a diameter, and a density, such that within a time interval of 24 hours at room temperature under a pressure of 1 bar at least 25% by weight of the vanillin and/or ethyl vanillin present in the composition is able to evaporate.

16. The device as claimed in claim 14, wherein the film has holes with a diameter, and a density, such that within a time interval of 24 hours at room temperature under a pressure of 1 bar at least 50% by weight of the vanillin and/or ethyl vanillin present in the composition is able to evaporate.

17. The device as claimed in claim 1, wherein said impermeable layer comprises aluminum foil.

18. The method of assisting a person in need thereof to regulate the craving to consume food by nasal or pulmonary uptake of at least one vanillin compound from a device as claimed in claim 1.

19. The method of assisting a person in need thereof to stimulate the release of serotonin in the central nervous system by nasal or pulmonary uptake of at least one vanillin compound from a device as claimed in claim 1.
